(19) United States
(12) Patent Application Publication
(10) Pub. No.: US 2024/0342474 A1
(43) Pub. Date: Oct. 17, 2024

(54) VAGUS NERVE STIMULATION PRE-SCREENING TEST

(71) Applicant: SetPoint Medical Corporation, Valencia, CA (US)

(72) Inventors: Jacob A. LEVINE, West Hempstead, NY (US); Ralph J. ZITNIK, Santa Barbara, CA (US)

(21) Appl. No.: 18/431,974

(22) Filed: Feb. 3, 2024

Related U.S. Application Data
(63) Continuation of application No. 17/506,597, filed on Oct. 20, 2021, now Pat. No. 11,890,471.
Application No. 17/506,597 is a continuation of application No. 16/103,873, filed on Aug. 14, 2018, now Pat. No. 11,173,307.
(60) Provisional application No. 62/545,284, filed on Aug. 14, 2017.

(51) Int. Cl. A61N 1/36; A61N 1/04; A61N 1/372; A61N 2/00; A61N 7/00; G01N 33/68

(52) U.S. Cl. CPC A61N 1/36053; G01N 33/6863; G01N 33/6869; A61N 1/0456; A61N 1/3606; A61N 1/372; A61N 2/006; A61N 2007/0026; G01N 2333/525; G01N 2333/535; G01N 2333/54; G01N 2333/5412; G01N 2333/5434; G01N 2333/545; G01N 2333/57; G01N 2800/52; G01N 2800/709; G01N 2800/7095

(57) ABSTRACT

Diagnostic screening tests that can be used to identify if a patient is a good candidates for an implantable vagus nerve stimulation device. One or more analyte, such as a cytokine or inflammatory molecule, can be measured from a blood sample taken prior to implantation of a vagus nerve stimulator to determine the patient's responsiveness to VNS for treatment of an inflammatory disorder.

### CROSS REFERENCE TO RELATED APPLICATIONS

This patent application is a continuation of U.S. patent application Ser. No. 17/506,597, filed Oct. 20, 2021, titled “VAGUS NERVE STIMULATION PRE-SCREENING TEST,” now U.S. Pat. No. 11,890,471, which is a continuation of U.S. patent application Ser. No. 16/103,873, filed Aug. 14, 2018, titled “VAGUS NERVE STIMULATION PRE-SCREENING TEST,” now U.S. Pat. No. 11,173,307, which claims priority to U.S. Provisional Patent Application No. 62/545,284, filed on Aug. 14, 2017, and titled “VAGUS NERVE STIMULATION PRE-SCREENING TEST,” which is herein incorporated by reference in its entirety.

The pre-screening methods and apparatuses described herein may be related to therapies and apparatuses such as those described in one or more of: the following pending U.S. patent applications: U.S. patent application Ser. No. 14/630,613, filed on Feb. 24, 2015, titled “VAGUS NERVE STIMULATION SCREENING TEST,” now U.S. Patent Publication No. 2015/0241447; U.S. patent application Ser. No. 12/620,413, filed on Nov. 17, 2009, titled “DEVICES AND METHODS FOR OPTIMIZING ELECTRODE PLACEMENT FOR ANTI-INFLAMATORY STIMULATION,” now U.S. Pat. No. 8,412,338; U.S. patent application Ser. No. 12/874,171, filed on Sep. 1, 2010, titled “PRESCRIPTION PAD FOR TREATMENT OF INFLAMMATORY DISORDERS,” Publication No. US-2011-0054569-A1; U.S. patent application Ser. No. 12/917,197, filed on Nov. 1, 2010, titled “MODULATION OF THE CHOLINERGIC ANTI-INFLAMMATORY PATHWAY TO TREAT PAIN OR ADDICTION,” Publication No. US-2011-0106208-A1; U.S. patent application Ser. No. 12/978,250, filed on Dec. 23, 2010, titled “NEURAL STIMULATION DEVICES AND SYSTEMS FOR TREATMENT OF CHRONIC INFLAMMATION,” now U.S. Pat. No. 8,612,002; U.S. patent application Ser. No. 12/797,452, filed on Jun. 9, 2010, titled “NERVE CUFF WITH POCKET FOR LEADLESS STIMULATOR,” now U.S. Pat. No. 8,886,339; U.S. patent application Ser. No. 13/467,928, filed on May 9, 2012, titled “SINGLE-PULSE ACTIVATION OF THE CHOLINERGIC ANTI-INFLAMMATORY PATHWAY TO TREAT CHRONIC INFLAMMATION,” now U.S. Pat. No. 8,788,034; and U.S. patent application Ser. No. 13/338,185, filed on Dec. 27, 2011, titled “MODULATION OF SIRTUINS BY VAGUS NERVE STIMULATION,” Publication No. US-2013-0079834-A1. Each of these patent applications is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference. The following is a list of publications that are incorporated by reference:
Andersson U, Tracey K. Reflex principles of immunological homeostasis. Annu Rev Immunol 2012; 30:313.
Bruchfeld A, et al. Whole blood cytokine attenuation by cholinergic agonists ex vivo and relationship to vagus nerve activity in rheumatoid arthritis. J Int Med 2010; 268:94.
Dake M. Chronic cerebrospinal venous insufficiency and multiple sclerosis: Hostory and background. Techniques Vase Intervent Radiol 2012; 15:94.
Ellrich J. Transcutaneous vagus nerve stimulation. Eur Neurological Rev 2011; 6:254-256.
Gao X, et al. Investigation of specificity of auricular acupuncture points in regulation of autonomic function in anesthetized rats. Autonomic Neurosc 1998; 88:109.
Huston J, et al. Transcutaneous vagus nerve stimulation reduces serum high mobility group box 1 levels and improves survival in murine sepsis. Crit Care Med 2007; 12:2762.
Koopman F, et al. Pilot study of stimulation of the cholinergic anti-inflammatory pathway with an implantable vagus nerve stimulation device in patients with rheumatoid arthritis. Arth Rheum 2012; 64 (10 suppl):S195.
M. L. Oshinsky, A. L. Murphy, H. Hekierski Jr., M. Cooper, B. J. Simon, Non-Invasive Vagus Nerve Stimulation as Treatment for Trigeminal Allodynia, PAIN (2014), doi: http://dx.doi.org/10.1016/j.pain.2014.02.009.
Peuker E. The nerve supply of the human auricle. Clin Anat 2002; 15:35.
Tekdemir I, et al. A clinico-anatomic study of the auricular branch of the vagus nerve and Arnold's ear-cough reflex. Surg Radiol Anat 1998; 20:253.
Yu L, et al. Low-level transcutaneous electrical stimulation of the auricular branch of the vagus nerve: A non-invasive approach to treat the initial phase of atrial fibrillation. Heart Rhythm 2013; 10: 428.
Zhao Y, et al. Transcutaneous auricular vagus nerve stimulation protects endotoxemic rat from lipopolysaccharide-induced inflammation. Evid Based Complement Alternat Med. 2012; 2012:627023. doi: 10.1155/2012/627023. Epub 2012 Dec. 29.

### FIELD

Embodiments of the invention relate generally to systems and methods for using vagus nerve stimulation for treatment, and more specifically to pre-screening methods and apparatuses for identifying patient that may respond to vagus nerve stimulation treatment for the treatment of inflammatory disorders.

### BACKGROUND

The vagus nerve mediates the inflammatory reflex, a mechanism the central nervous system utilizes to regulate innate and adaptive immunity. The afferent arm of the reflex senses inflammation both peripherally and in the central nervous system, and down-regulates the inflammation via efferent neural outflow. The efferent arm of this reflex has been termed the “cholinergic anti-inflammatory pathway” (CAP). The reflex serves as a physiological regulator of inflammation by responding to environmental injury and pathogens with an appropriate degree of immune system activation (Andersson, 2012). CAP activation can also be harnessed to reduce pathological inflammation. Activating the CAP chronically using electrical neurostimulation of the vagus nerve is a feasible means of treating diseases characterized by excessive and dysregulated inflammation.

Although vagus nerve stimulation has been shown to be effective in some patients, the efficacy of the treatment may vary, particularly at low to moderate stimulation levels. Responders are those patient's that response strongly to vagus nerve stimulation, particularly for the treatment of inflammatory disorders, including those described above, and incorporated by reference. However, there are some patients for whom vagus nerve stimulation alone, and particularly electrical vagus nerve stimulation, may not be sufficient. These patients may be referred to as non-responders or low responders. It would be particularly helpful to be able to a priori determine if a particular patient will respond well to a vagus nerve stimulation. Described herein are methods and apparatuses that may be used to identify, e.g., responders from non-responders and/or low-responders.

### SUMMARY OF THE DISCLOSURE

The present invention relates generally to systems and methods for using vagus nerve stimulation for treatment, and more specifically to screening tests for identifying suitable patients for vagus nerve stimulation treatment.

In general, described herein are method for screening a patient for responsiveness to vagus nerve stimulation by challenging an extracted blood sample from the patient (either fresh or frozen). Also descried herein are alternatively or additional methods for determining if a patient will respond to vagus nerve stimulation by comparing patient levels of a biomarker before and after vagus nerve stimulation. Surprisingly, techniques for examining extracted blood in the absence of vagus nerve stimulation may predict how well a patient may respond to vagus nerve stimulation, including in particular, vagus nerve stimulation to treat an inflammatory disorder using an implanted vagus nerve stimulator.

For example, described herein are methods of treating a patient having an inflammatory disorder with vagus nerve stimulation. In some variations, these methods may be methods of determining if a patient will respond (e.g., is a responder) or will not respond (e.g., is a non-responder) to vagus nerve stimulation and therefore should or should not have a vagus nerve stimulation device implanted. For example, a method of treating a patient having an inflammatory disorder with vagus nerve stimulation may include: challenging a sample of blood with a first concentration of toxin, wherein the sample is taken from the patient prior to implantation of a vagus nerve stimulator; challenging a second sample of blood with a second concentration of toxin that is at least 2 times (e.g., 3×, 4×, 5×, 6×, 7×, 8×, 9×, 10×, 15×, 20×, 25×, 30×, 50×, 100×, etc.) the first concentration, wherein the second sample is taken from the patient prior to implantation of the vagus nerve stimulator; and implanting the vagus nerve stimulator to treat the patient for the inflammatory disorder when an amount of an inflammatory cytokine released in response to the second concentration of toxin is greater than an amount of the inflammatory cytokine released in response to the first concentration of toxin by a threshold.

Any of these methods may include determining a ratio of the amount of the inflammatory cytokine released in response to the second concentration of toxin and the amount of the inflammatory cytokine released in response to the first concentration, and may compare this ratio to a threshold ratio (e.g., a cutoff threshold). For example, the threshold ratio for the change in inflammatory cytokine release due to increasing toxin amount may be, e.g., 50, 55, 60, 65, 70, 75, 80, 85, 90, 95, 100, etc. In some variations the threshold ratio is 60 (e.g., a patient is a good candidate if the ratio is greater than 60).

Any of the methods described herein may include displaying an indication that the patient is a responder or a non-responder based on the amount of the inflammatory cytokine released in response to the second concentration of toxin compared to the amount of the inflammatory cytokine released in response to the first concentration of toxin (including based on the ratio of the inflammatory cytokine release at different levels of toxin).

The methods described herein may include measuring the amount of the inflammatory cytokine that is released in response to the first concentration and measuring the amount of the inflammatory cytokine that is released in response to the second concentration.

As described in greater detail below, the toxin may be an endotoxin, including Lipopolysaccharide (LPS).

Although a first and a second level of toxin are described, one or more additional toxin levels may also be described, such as a third or fourth toxin challenge. For example, the method may include challenging a third sample of blood with a third concentration of toxin that is at least 2 times the second concentration, wherein the third sample is taken from the patient prior to stimulation of the patient's vagus nerve and wherein implanting the vagus nerve stimulator comprises implanting the vagus nerve stimulator when an amount of an inflammatory cytokine released in response to the third concentration of toxin is greater than an amount of the inflammatory cytokine released in response to the first and second concentrations of toxin by the threshold.

In some variations, challenging the second sample of blood with the toxin at the second concentration may comprises challenging with a concentration that is at least 10 times the first concentration.

In general, the first sample of blood and the second sample of blood may be portions of a master sample taken from the patient. Thus, the same blood sample may be challenged with different levels of toxin.

The inflammatory cytokine may be one or more of: tumor necrosis factor (TNF), interleukin-1 (IL-1), interleukin-6 (IL-6), interleukin-12 (IL-12), interleukin-18 (IL-18), inflammsome, interferon gamma, and granulocyte-macrophage colony stimulating factor. In some variations, the inflammatory cytokine may be TNF.

For example, a method of treating a patient having an inflammatory disorder with vagus nerve stimulation may include: obtaining a sample of blood taken from the patient prior to implantation of a vagus nerve stimulator; challenging the sample of blood with LPS at a first concentration of LPS; measuring a level of an inflammatory cytokine that is released in response to the LPS challenge at the first concentration of LPS; challenging the sample of blood with LPS at a second concentration of LPS that is at least 5 times the first concentration of LPS; measuring a level of the inflammatory cytokine that is released in response to the LPS challenge at the second concentration of LPS; determining a response curve based on the level of the inflammatory cytokine that is released in response to the LPS challenges at the first concentration of LPS and the second concentration of LPS; and implanting the vagus nerve stimulation when the determined response is greater than a cutoff threshold.

The second concentration of LPS may be at least 10 times the first concentration of LPS. The method may include challenging the sample of blood with LPS at a third concentration of LPS that is at least 5 times the second concentration of LPS, and wherein the response curve is determined based on the level of the inflammatory cytokine that is released in response to the LPS challenges at the first concentration of LPS, the second concentration of LPS, and the third concentration of LPS. The inflammatory cytokine may be selected from the group consisting of TNF, IL-1, IL-6, IL-12, IL-18, inflammsome, interferon gamma, and granulocyte-macrophage colony stimulating factor. For example, the inflammatory cytokine nat be TNF. The step of determining the response curve may include calculating a slope of the response curve. The method may include comprising comparing the determined response curve to the cutoff threshold by comparing a slope of the response curve to the cutoff threshold. Implanting may comprises determining whether a slope of the response curve is greater than the cutoff threshold.

The methods (and apparatuses for performing them) described herein may include in vitro methods for screening a patient for responsiveness to vagus nerve stimulation to treat an inflammatory disorder. For example, a method may include: challenging a sample of blood with a first concentration of toxin, wherein the sample is taken from the patient prior to stimulation of the patient's vagus nerve; challenging a second sample of blood with a second concentration of toxin that is at least 2 times the first concentration, wherein the second sample is taken from the patient prior to stimulation of the patient's vagus nerve; and outputting an indication that the patient is a responder or a non-responder for vagus nerve stimulation based on the amount of inflammatory cytokine released in response to the second concentration of toxin compared to the inflammatory cytokine released in response to the first concentration of toxin.

Any of these methods may include determining if the patient is a responder for vagus nerve stimulation by determining that a ratio of the amount of the inflammatory cytokine released in response to the second concentration of toxin and the amount of the inflammatory cytokine released in response to the first concentration of toxin is greater than a threshold ratio. For example, the threshold ratio may be greater than 60. Any of these methods may include challenging the sample of blood with the toxin at a third concentration that is at least 2 times the second concentration and measuring a level of the inflammatory cytokine that is released in response to the third concentration, wherein outputting the indication that the patient is a responder for vagus nerve stimulation treatment to treat an inflammatory disorder is based on the amount of inflammatory cytokine released in response to the third concentration of toxin relative to the response to the first and second concentrations of toxin.

Challenging the sample of blood with the toxin at a second concentration may comprise challenging with a concentration that is at least 10 times the first concentration.

An in vitro method for screening a patient for responsiveness to vagus nerve stimulation may include: obtaining a sample of blood taken from the patient prior to stimulation of the patient's vagus nerve; challenging the sample of blood with LPS at a first concentration of LPS; measuring a level of an inflammatory cytokine that is released in response to the LPS challenge at the first concentration of LPS; challenging the sample of blood with LPS at a second concentration of LPS that is at least 5 times the first concentration of LPS; measuring a level of the inflammatory cytokine that is released in response to the LPS challenge at the second concentration of LPS; and determining a response curve based on the level of the inflammatory cytokine that is released in response to the LPS challenges at the first concentration of LPS and the second concentration of LPS; comparing the determined response curve to a cutoff; and determining whether the patient is suitable for vagus nerve stimulation based on the comparison of the determined response curve to the cutoff.

The second concentration of LPS may be at least 10 times the first concentration of LPS. The method may also include challenging the sample of blood with LPS at a third concentration of LPS that is at least 5 times the second concentration of LPS, and wherein the response curve is determined based on the level of the inflammatory cytokine that is released in response to the LPS challenges at the first concentration of LPS, the second concentration of LPS, and the third concentration of LPS. The inflammatory cytokine may be selected from the group consisting of TNF, IL-1, IL-6, IL-12, IL-18, inflammsome, interferon gamma, and granulocyte-macrophage colony stimulating factor (e.g., TNF). The step of determining the response curve may include calculating a slope of the response curve. Comparing the determined response curve to the cutoff may include comparing the slope of the response curve to the cutoff.

Determining whether the patient is suitable for vagus nerve stimulation may include determining whether the slope of the response curve is greater than the cutoff.

Also described herein are methods that examine the levels of one or more biomarker(s) before and after an intentional vagal nerve stimulation (e.g., a mechanical and/or electrical stimulation of the vagus nerve). For example, a method for screening a patient for responsiveness to vagus nerve stimulation may include: measuring a baseline level of C-reactive protein (CRP) in a sample of blood taken from the patient; intentionally stimulating the vagus nerve (e.g., mechanically and/or electrically); measuring a post-stimulation level of CRP in a sample of blood taken after the vagus nerve has been stimulated; comparing the post-stimulation level of CRP to the baseline level of CRP; and indicating if the patient is a suitable candidate for an implantable vagus nerve stimulation device based on the comparison of the post-stimulation level of CRP to the baseline level of CRP.

Indicating may comprise indicating that the patient is a suitable candidate for the implantable vagus nerve stimulation device when the post-stimulation level of CRP increases by 5% or more.

The vagus nerve may be stimulated in any appropriate manner, including noninvasively, with transcutaneous electrical stimulation, by mechanical stimulation, stimulating the auricular branch of the vagus nerve, stimulating a cervical portion of the vagus nerve, or combinations of these. For example, any of these methods may include: introducing an electrode intravascularly via percutaneous puncture; and positioning the electrode at a cervically located blood vessel proximate the vagus nerve. In some variations the method may include introducing an electrode into the carotid sheath; and positioning the electrode within the carotid sheath such that the electrode is proximate the vagus nerve. The methods may include: placing an electrode of a transcutaneous electrical nerve stimulation device on the patient's skin over the cervical vagus nerve.

For example, a method of treating a patient having an inflammatory disorder with vagus nerve stimulation may include: measuring a baseline level of C-reactive protein (CRP) in a sample of blood taken from the patient; stimulating the patient's vagus nerve; measuring a post-stimulation level of CRP in a sample of blood taken after the vagus nerve has been stimulated; comparing the post-stimulation level of CRP to the baseline level of CRP; and implanting a vagus nerve stimulator if the patient is a suitable candidate for an implantable vagus nerve stimulation device based on the comparison of the post-stimulation level of CRP to the baseline level of CRP.

A method for screening a patient for responsiveness to vagus nerve stimulation may include: measuring a baseline level of a cytokine and one or more of lymphocytes and monocytes in a sample of blood taken from the patient; stimulating the vagus nerve; measuring a post-stimulation level of the cytokine and the one or more of lymphocytes and monocytes in a sample of blood taken after the vagus nerve has been stimulated; comparing the post-stimulation level of the cytokine and the one or more of lymphocytes and monocytes to the baseline level of the cytokine and the one or more of lymphocytes and monocytes; and indicating if the patient is a suitable candidate for an implantable vagus nerve stimulation device based on the comparison of the post-stimulation levels of the cytokine and the one or more of lymphocytes and monocytes to the baseline levels of the cytokine and the one or more of lymphocytes and monocytes.

The cytokine may comprise interleukin 7 (IL-7) and the one or more of lymphocytes and monocytes comprise CD3− CD19+ cells (e.g., cells recognized by the well-known CD3 antibody recognize some human T lymphocyte, and CD19 antibody recognizes human B lymphocytes).

Indicating may comprise indicating a patient is a responder if the patient indicated an increase or a decrease of 10% or less (e.g., 10% or less, 9% or less, 8% or less, 7% or less, 6% or less, 5% or less, 4% or less, 3% or less, 2% or less, 1% or less, etc., less than 10%, etc.) in the cytokine and the one or more of lymphocytes and monocytes compared to baseline. Indicating may comprise indicating that the patient is a non-responder if the cytokine and the one or more of lymphocytes and monocytes exhibited a greater than 10% (e.g., greater than 11%, greater than 12%, greater than 13%, greater than 14%, greater than 15%, greater than 20%, greater than 25%, etc.) decrease in CD3−, CD19+(e.g., B-lymphocytes) cell populations and a greater than 10% decrease in IL-7 levels compered to baseline. The method vagus nerve may be stimulated noninvasively, and/or with transcutaneous electrical stimulation, and/or by mechanical stimulation, and/or by stimulating the auricular branch of the vagus nerve, and/or by stimulating a cervical portion of the vagus nerve, etc. For example, the method may include introducing an electrode intravascularly via percutaneous puncture; and positioning the electrode at a cervically located blood vessel proximate the vagus nerve. The method may include introducing an electrode into the carotid sheath; and positioning the electrode within the carotid sheath such that the electrode is proximate the vagus nerve. The method may include: placing an electrode of a transcutaneous electrical nerve stimulation device on the patient's skin over the cervical vagus nerve.

Also described herein are methods of treating a patient having an inflammatory disorder with vagus nerve stimulation, the method comprising: measuring a baseline level of a cytokine and one or more of lymphocytes and monocytes in a sample of blood taken from the patient; stimulating the vagus nerve; measuring a post-stimulation level of the cytokine and the one or more of lymphocytes and monocytes in a sample of blood taken after the vagus nerve has been stimulated; comparing the post-stimulation level of the cytokine and the one or more of lymphocytes and monocytes to the baseline level of the cytokine and the one or more of lymphocytes and monocytes; and implanting a vagus nerve stimulator if the patient is a suitable candidate for an implantable vagus nerve stimulation device based on the comparison of the post-stimulation level of the cytokine and the one or more of lymphocytes and monocytes compared to the baseline level of the cytokine and the one or more of lymphocytes and monocytes.

For example, described herein are methods for screening a patient for responsiveness to vagus nerve stimulation comprising all of some of the following steps: obtaining a sample of blood taken from the patient prior to application of vagal nerve stimulation of the patient; challenging the sample of blood with an agent that evokes an immune response, e.g., a toxin or particularly an endotoxin, such as Lipopolysaccharides (LPS), at a first concentration; measuring a level of an inflammatory cytokine that is released in response to the challenge at the first concentration; challenging the sample of blood with the same or a different toxin (and particularly an endotoxin such as LPS) at a second concentration that is higher than the first concentration (e.g., at least twice, 3×, 4×, 5×, 6×, 7×, 8×, 9×, 10×, 11×, 15×, 20×, 25×, 30×, etc.); measuring a level of the inflammatory cytokine that is released in response to the second challenge at the second concentration; and determining if the patient is suitable for vagus nerve stimulation based on the response to the second challenge relative to the first challenge. For example, determining if the patient is suitable for vagus nerve stimulation may be based on a response curve of the level of the inflammatory cytokine that is released in response to the challenges at the first concentration (e.g. of LPS) and the second concentration (e.g., of LPS), and comparing the determined response curve to a threshold.

For example, when the challenge is an LPS challenge, the second concentration of LPS may be at least 10 times the first concentration of LPS.

Any of these methods may also include challenging the sample of blood with a third concentration of toxin (e.g., endotoxin such as LPS) that is at twice (e.g., at least 2×, 3×, 4×, 5×, 6×, 7×, 8×, 9×, 10×, 11×, 12×, 15×, 20×, 25×, 30×, etc.) the second concentration, and wherein the fitness for vagal nerve stimulation is determined based on the level of the inflammatory cytokine that is released in response to the third challenges relative to the first and/or second challenge concentrations.

In any of the methods described herein, the inflammatory cytokine examined from the blood sample may be one or more of: TNF, IL-1, IL-6, IL-12, IL-18, inflammsome, interferon gamma, and granulocyte-macrophage colony stimulating factor. The inflammatory cytokine may be TNF.

In any of these example, a response curve may be determined. For example, determining a response curve may include calculating a slope of the response curve. The determined response curve may be compared to a cutoff, e.g., by comparing the slope of the response curve to the cutoff.

Determining whether the patient is not suitable for vagus nerve stimulation may include determining whether the slope of the response curve is less than the cutoff.

In general, the methods described herein may be performed on blood ex vivo; the blood sample may be withdrawn from the patient and stored (e.g., frozen, etc.) or used fresh, and challenged over time or simultaneously with multiple levels of toxin (e.g., endotoxin). This may allow the patient to be screened without any associated risk to the patient, prior to any surgical intervention, including implanting a neurostimulator (vagus stimulator).

For example, venous blood may be drawn from the patient, an anticoagulant (e.g., heparin) added, and the blood may be aliquoted to tubes. Thereafter a toxin (endotoxin, such as LPS) may be mixed with the blood to final concentrations, e.g., of 0, 1, 10, and 100 ng/mL; the blood may be incubated, e.g., for 4 hr at 37° C. The plasma may be separated and an immune response (e.g., TNF level) measured, e.g., by ELISA.

Also described herein are methods of screening patient based on comparing a response of an inflammatory marker (e.g., C-reactive protein, CRP) before and after vagal nerve stimulation. Vagal nerve stimulation (VNS) may be performed anywhere on the vagus nerve, or and/or an associated nucleus. For example, VNS may be performed by transcranial stimulation to nucleus tractus solitarius (NTS), dorsal motor nucleus of the vagus (DMV), or Locus coeruleus (LC), for example.

For example, a method for screening a patient for responsiveness to vagus nerve stimulation may include: measuring a baseline level of CRP in a sample of blood taken before the vagus nerve has been stimulated; stimulating the vagus nerve after the step of measuring the baseline level of CRP; measuring a post stimulation level of CRP in a sample of blood taken after the vagus nerve has been stimulated; comparing the post stimulation level of CRP to the baseline level of CRP; and determining whether the patient is a suitable candidate for an implantable vagus nerve stimulation device based on the post-stimulation level of CRP relative to the baseline level of CRP. The stimulation of the vagus nerve may be done noninvasively. The vagus nerve stimulation may be done with transcutaneous electrical stimulation. The vagus nerve stimulation may be done with mechanical stimulation. The step of stimulating the vagus nerve may comprise stimulating the auricular branch of the vagus nerve. The stimulation of the vagus nerve may comprise stimulating a cervical portion of the vagus nerve.

For example, the method may include introducing an electrode intravascularly via percutaneous puncture; and positioning the electrode at a cervically located blood vessel proximate the vagus nerve.

The method may include introducing an electrode into the carotid sheath; and positioning the electrode within the carotid sheath such that the electrode is proximate the vagus nerve. Alternatively or additionally, the method may include placing an electrode of a transcutaneous electrical nerve stimulation device on the patient's skin over the cervical vagus nerve.

Any of the pre-screening methods described herein may also include one or more of: administration of nicotinic agonist, for example, a nicotinic a7 agonists, and/or one or more cholinesterase inhibitor(s).

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-1C illustrate various embodiments of a mechanical stimulator.

FIG. 2 illustrates an embodiment of a transcutaneous electrical nerve stimulation device.

FIGS. 3A-3C illustrate various embodiments of minimally invasive electrical stimulation of the vagus nerve.

FIG. 3D illustrates an embodiment of minimally invasive mechanical stimulation of the vagus nerve.

FIGS. 4A-4C illustrate that that there was no obvious or significant difference in LPS-induced TNF levels under various LPS concentrations between European League Against Rheumatism (EULAR) criteria responders and nonresponders in the two cohorts of patients tested.

FIG. 5 illustrates LPS dose response curves for responders and nonresponders using blood samples before vagus nerve stimulation.

FIG. 6 illustrates LPS dose response curves for responders and nonresponders using blood samples before vagus nerve stimulation.

FIG. 7 illustrates the slopes of the LPS dose response curves for responders and non-responders to 100 ng/ml LPS.

FIG. 8 illustrate the slopes of the LPS dose response curves for responders and non-responders to 10 ng/ml LPS.

FIG. 9 illustrates the change in CRP levels after vagus nerve stimulation comparing responders and nonresponders.

FIG. 10 illustrates the change in CRP levels after vagus nerve stimulation for responders and nonresponders (based on the change from baseline).

FIG. 11 illustrates another example of the change in CRP levels after vagus nerve stimulation for responders and nonresponders (based on the change from baseline).

FIG. 12 is a table that shows changes in cell populations and IL-7 levels between screening day and after implantation of the vagus nerve stimulation device.

FIG. 13 is a table that shows changes in cell populations and IL-7 levels between screening day and after implantation of the vagus nerve stimulation device.

### DETAILED DESCRIPTION

In general, described herein are methods and apparatuses for determining, e.g., by a blood test, if a subject (e.g., a mammalian, including human, patient) will benefit and/or respond to electrical and/or mechanical vagus nerve stimulation (VNS) in order to treat an inflammatory disorder.

In particular, described herein are methods and apparatuses (devices, systems, assays, etc.) for taking a blood sample from a patient and performing one or more procedures on the blood sample, without having to subject the patient to any stimulation. This may allow for the identification of patients that may benefit from the therapy (or may require additional or adjunctive therapies or modifications in the level of therapy provided) prior to providing any therapy to the patient. Since the methods may be performed just on an ex vivo sample removed from the patient's body, the patient may be spared any risk, complications, and/or discomfort due to stimulation.

Described herein are three variations of the ex vivo methods for determining if a patient will respond to vagus nerve stimulation. For example, described herein are methods for treatment of Rheumatoid Arthritis (RA) by activating the CAP using electrically active implantable medical devices; these methods may include first confirming that the patient is a candidate for treatment using any of the methods described herein.

For example, a study of vagus nerve stimulation (VNS) using an electrically active surgically implanted medical device in 8 patients with active rheumatoid arthritis (RA) was performed at 4 investigative centers. The device used an implanted helical coiled cuff lead to deliver electrical stimulation from an implanted pulse generator. After 6 weeks of stimulation, clinically significant improvements were seen in 6 of 8 patients as assessed by standard RA efficacy outcome measures such as the Disease Activity Score (DAS). These results provide proof-of-concept for the therapeutic use of VNS in RA as an alternative to small molecule and biological agent therapy. However, while the majority of the patients in the study responded, 2 of 8 did not have a meaningful clinical response. In order for VNS to be more successful as a therapy, it would be desirable to use a diagnostic screening test to preoperatively predict likelihood of clinical response to an implant, thus sparing patients who are unlikely to respond from undergoing an unnecessary surgical procedure. The diagnostic screening tests described herein may utilize various techniques, such as mechanical and electrical stimulation to stimulate the vagus nerve noninvasively and/or in some cases an iv vitro toxin challenge, to determine if a patient will respond to treatment prior to implantation of the electrical stimulation.

For example, in some variations, the effectiveness of CAP Activation may be determined by stimulation of the auricular branch of the vagus verve prior to implantation. Sensory fibers of the Auricular Branch of the Vagus Nerve (ABVN) innervate the skin of the cymba concha of the external ear. These fibers may provide afferent input via the superior ganglion of the vagus to the brainstem nucleus of the solitary tract (NST). The neurons of the NST then project to efferent neurons originating in the dorsal nucleus and nucleus ambiguous of the vagus nerve, which then in turn project within the vagus nerve to the visceral organs.

This neuroanatomical pathway may provide a potential mechanism for non-invasive activation of the CAP by induction of efferent vagal outflow through stimulation of the afferent ABVN pathway using mechanical or electrical stimulation of the skin of the cymba concha. The ABVN is particularly suitable for noninvasive stimulation because the nerves are located relatively close to the surface of the skin. Similarly, other portions of the vagus nerve that are similarly situated close to the patient's skin may be used for noninvasive stimulation. For example, the nerve may be located less than about 2, 1, 0.5 or 0.25 cm from the surface of the patient's skin. The terms “about”, “approximately” and the like can mean within 10%, 20%, or 30%.

The presence of such a functional reflex pathway (immune reflex pathway) in the ABVN is suggested by the following observations: (1) the “Arnold's reflex” occurs in approximately 2% of the population and results in coughing or gagging in response to mechanical stimulation of the ear canal; (2) ABVN electrical stimulation in rats resulted in parasympathetic vagally-mediated reductions in heart rate and blood pressure, and increased intra-gastric pressure. This response could be abrogated with atropine; (3) ABVN electrical stimulation in dogs reverses the acute right atrial remodeling and reduction in threshold for inducible atrial fibrillation caused by rapid right atrial pacing; and this protective effect of ABVN could be eliminated by bilateral transection of the vagus in its upper thoracic segment; (4) the ability of ABVN stimulation to activate the CAP and inhibit systemic inflammation in rodents. In a rodent systemic endotoxemia model, transcutaneous electrical ABVN stimulation was compared to VNS delivered using a surgically placed cervical electrode. While cervical VNS was the most effective, ABVN stimulation caused significant reductions in circulating tumor necrosis factor (TNF), Interleukin (IL)-1 beta, and IL-6, thus demonstrating that the CAP can be effectively activated by transcutaneous auricular stimulation.

Described herein are methods and apparatuses for using the CAP activation as a diagnostic screening test by applying a short duration stimulation of the skin of the external ear, for example either mechanically or with an electric current. The stimulation duration can be between about 1 second to 24 hours and can be applied either continuously or intermittently throughout the duration. In some embodiments, the duration is less than about 1, 5 10, 20, 30, or 60 seconds. In some embodiments, the duration is less than about 1, 2, 3, 4, 5, 10, 15, 20, 30, 40, 50, or 60 minutes. In some embodiments, the duration is less than about 1, 2, 3, 4, 5, 6, 12, 18, or 24 hours.

The non-invasive stimulation may include mechanical stimulation of a body region such as the subject's ear. Examples of non-invasive stimulation devices are described in U.S. Publication No. 2008/0249439 to Tracey et al., which is herein incorporated by reference in its entirety. In particular, the cymba conchae region of their ear may be stimulated. The non-invasive stimulation may comprise mechanical stimulation between about 1 and 500 Hz, or 30 Hz and 500 Hz, or 50 Hz and 500 Hz. In some variations the stimulation is transcutaneous stimulation applied to the appropriate body region (e.g., the ear). For example, transcutaneous stimulation may be applied for an appropriate duration (e.g., less than 24 hours to less than 1 hour, less than 60 minutes to less than 1 minute, less than 60 seconds to less than 1 second, etc.), at an appropriate intensity and frequency. Stimulation that does not significantly affect cardiac measures may be particularly desirable, and the stimulation may be limited to such a range, or may be regulated by cardiac feedback (e.g., ECG, etc.).

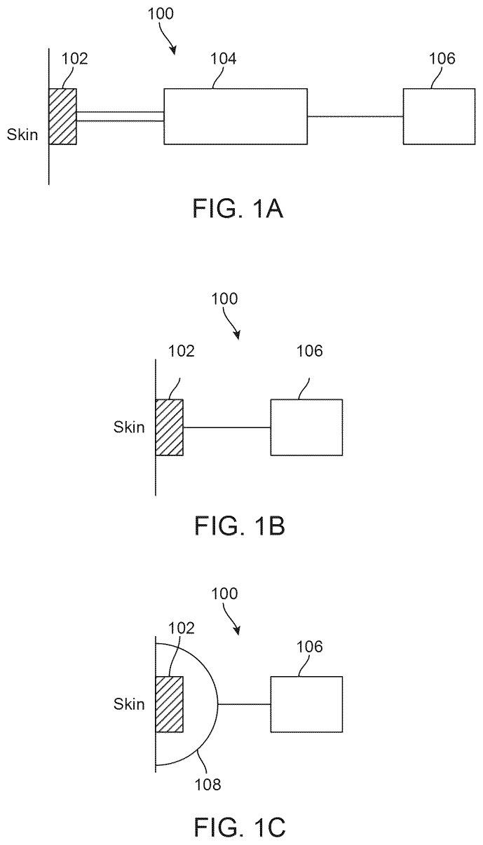

Also described herein are devices for non-invasively mechanically stimulating a subject's inflammatory reflex, as illustrated in FIGS. 1A-1C. These mechanical stimulation devices 100 may include an actuator 102, such as a movable distal tip region that is configured to mechanically stimulate at least a portion of a subject's ear, a handle 104, and a driver 106 configured to move the distal tip region between about 50 and 500 Hz. In some variations, the stimulation devices are part of a system including a stimulation device. In some embodiments, the actuator 102 can be directly adhered to the patient's skin, thereby removing the need for a handle. The actuator 102 can be coated with an adhesive or can be integrated into an adhesive pad or pod 108.

A stimulation device may include a controller configured to control the driver so that it applies stimulation within stimulation parameters. For example the controller (which may be part of the driver, or may be separate from the driver) may control the intensity (e.g., force, displacement, etc.), the timing and/or frequency (e.g., the frequency of repeated pulses during a stimulation period, the stimulation duration during the period of stimulation, the duration between stimulation periods, etc.), or the like. In some variations the controller is pre-programmed. In some variations, the controller receives input. The input may be control input (e.g., from a physician or the patient) that modifies the treatment. In some variations the stimulator device includes a therapy timer configured to limit the duration of stimulation. For example, the controller may be configured to limit the period of stimulation to less than 10 minutes, less than 5 minutes, less than 3 minutes, less than 1 minute, etc.

Any appropriate driver may be used. For example, the driver may be a motor, voice (or speaker) coil, electromagnet, bimorph, piezo crystal, electrostatic actuator, and/or rotating magnet or mass. For example, in some variations the driver is a mechanical driver that moves an actuator against the subject's skin. Thus, an actuator may be a distal tip region having a diameter of between about 35 mm and about 8 mm.

In some variation the stimulator includes a frequency generator that is in communication with the driver. Thus the driver may control the frequency generator to apply a particular predetermined frequency or range of frequencies to the actuator to non-invasively stimulate the subject.

The stimulator devices described herein may be hand-held or wearable. For example, also described herein are wearable device for non-invasively stimulating a subject's inflammatory reflex. These stimulator the devices may include an actuator configured to mechanically stimulate a subject's cymba conchae, a driver configured to move the distal tip region between about 1 and 500 Hz, or 30 Hz and 500 Hz, or 50 Hz and 500 Hz, and an ear attachment region configured to secure to at least a portion of a subject's ear.

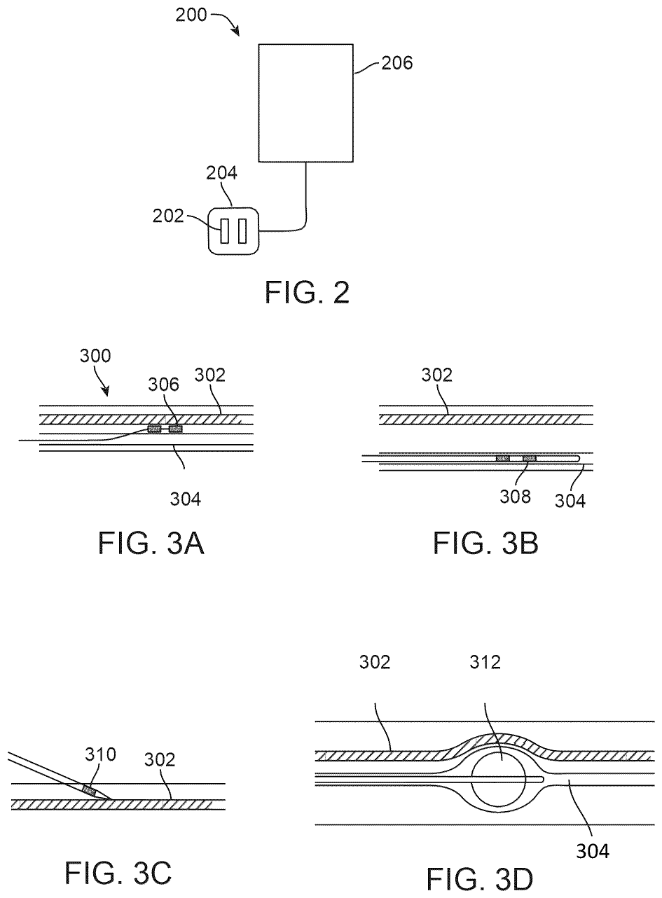

Transcutaneous electrical nerve stimulation (TENS) can be provided by an electrical stimulation device 200 having at least one electrode 202 that can be placed on the patient's skin, as illustrated in FIG. 2. The electrode 202 can be integrated into an adhesive patch or pad 204 that can be adhered to the patient's skin. A lead can connect the electrode with the housing 206 of the device. Alternatively, the housing can also be integrated with the adhesive patch or pad. A control or signal generator can deliver the electrical signal stimulus through the electrode. The signal amplitude can be between about 0.05 to 10 mA, or 0.05 to 15 mA, or 0.05 to 20 mA, or 0.05 to 25 mA, or 0.05 to 50 mA. The pulse width can be between about 100 and 1,000 μS. The pulse frequency can be between about 1 and 50 Hz, and the stimulus duration can be between about 1 second and 24 hours.

The electrical parameters can be similar when the electrical stimulation is provided by an electrode that has been inserted into the patient through minimally invasive techniques as described herein. For example, the signal amplitude can be between about 0.05 to 5 mA, or 0.05 to 10 mA, or 0.05 to 15 mA, 0.05 to 20 mA, or 0.05 to 25 mA or 0.05 to 50 mA.

In some variations a method for treating a patient and/or determining if a patient is a candidate for treatment may include CAP activation by stimulation of the cervical vagus nerve. These methods may include acutely invasive techniques. For example, temporary electrical stimulation of the cervical vagus nerve for screening purposes can be performed using a variety of devices. A device can include a non-cuffed lead 306 placed percutaneously through a blood vessel 304 such as the internal jugular vein on or near the vagus nerve 302 within the carotid sheath 300, as illustrated in FIG. 3A. Alternatively, as shown in FIG. 3B, a standard percutaneous catheter-directed intravascular electrode 308 can be positioned within the internal jugular 304 or other nearby blood vessel, in close anatomical apposition to the vagus nerve 302, with electrical stimulation delivered trans-vascularly. Other electrode or lead configurations, such as needle electrodes 310, can also be used to temporarily stimulate the vagus nerve 302, as shown in FIG. 3C.

Alternatively, direct mechanical stimulation of the cervical vagus nerve by physical manipulation has been demonstrated to activate the CAP in the rodent endotoxemia model. Angioplasty of the internal jugular vein may be performed using percutaneously inserted balloon-tipped angioplasty catheters. Transmural pressure can be exerted internally within the internal jugular vein 304 using such balloon catheters 312 in order to mechanically stimulate the nearby cervical vagus nerve 302 and activate the CAP, as shown in FIG. 3D. Other mechanical stimulators or expanders can also be used to provide mechanical stimulation.

Alternatively, the cervical vagus nerve can also be stimulated using intravascular or transcutaneous ultrasound, transcutaneous magnetic energy, transcutaneous RF energy, and transcutaneous electric stimulation.

The CAP can be activated in the diagnostic screening test described herein by short duration (1 second to 24 hour) stimulation of the cervical vagus nerve using the above methods, either mechanically or with an electric current.

Furthermore, in some variations, CAP activation specific to a patient may be examined using a bioassay performed on one or more peripheral blood samples. The degree of CAP activation induced by the stimulation methods described above can be measured in a diagnostic screening test using several biological activity assays performed on either serum samples, supernatants from whole blood samples cultured in vitro in the presence of cytokine release stimulators, or supernatants from separated cellular constituents of whole blood cultured in vitro in the presence of cytokine release stimulators. Alternatively or additionally, the assay may be based on tissue samples or other biological fluids. The reduction or increase in these mediators that is observed between a pre-stimulus measurement and a post-stimulus measurement, and/or between stimulation at different levels, may be indicative of the responsiveness of the patient to CAP activation, and may predict the response to a permanently implanted VNS system. For example, a predetermined change in level or concentration of a mediator, cytokine, or analyte from a baseline level or concentration before stimulation may indicate responsiveness of the CAP to stimulation and suitability of the patient for an implanted VNS system. Alternatively, the reduction or increase in inducible release of a cytokine, mediator, signaling molecule, or other analyte in a cell-based assay can also be used. In some embodiments, the predetermined change is a reduction of at least 10, 20, 30, 40 or 50%. In some embodiments, the predetermined change is an increase of at least 10, 20, 30, 40 or 50%.

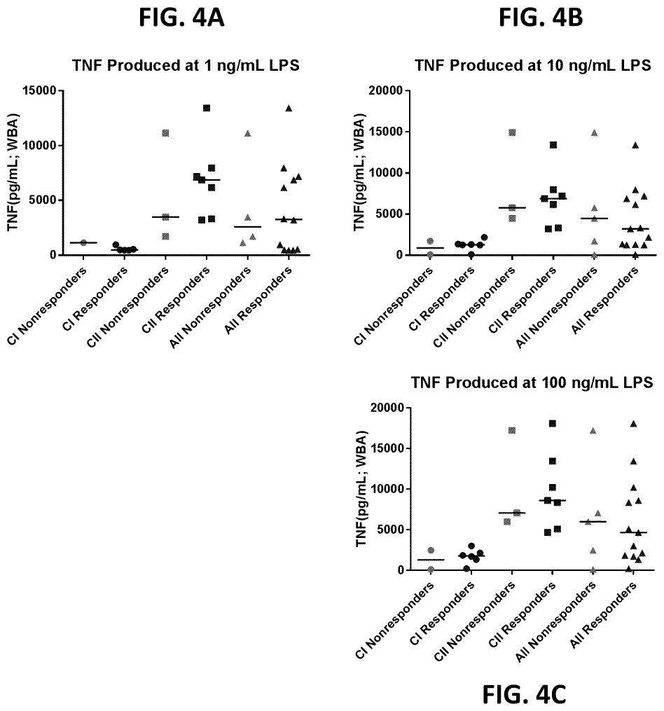

For example, one bioassay measures endotoxin, e.g., lipopolysaccharide (LPS), induced TNF levels in whole blood samples taken during the screening day before implantation of the VNS stimulator or before any stimulation of the vagus nerve. FIGS. 4A-8 illustrate one set of experiments characterizing an in vitro assay in which blood samples from both responders and non-responders (taken before implantation) were compared. To perform the whole blood assay (WBA), venous blood was drawn from each patient and dispensed with heparin into separate tubes on a screening day (e.g., day −21). LPS (endotoxin) was mixed with the blood to final concentrations of 0, 1, 10, and 100 ng/mL. Blood was incubated for 4 hours at 370 Celsius. Plasma was then separated from the whole blood and TNF levels were measured by ELISA. On Day −14, seven days after the screening day, the patients were then implanted with a cervical vagus nerve stimulation device for treatment of inflammation caused by rheumatoid arthritis, and on Day 0, two weeks after implantation the vagus nerve was stimulated with the implanted device and subsequently stimulated according to a prescribed therapy regimen. The patients were characterized as responders or nonresponders based on their response to the VNS therapy. The screening test data was then analyzed.

FIGS. 4A-4C illustrate that there was no obvious or significant difference in LPS-induced TNF levels under various LPS concentrations between European League Against Rheumatism (EULAR) criteria responders and nonresponders in the two cohorts of patients tested. No difference is seen because the amount of TNF produced appears to be highly patient specific.

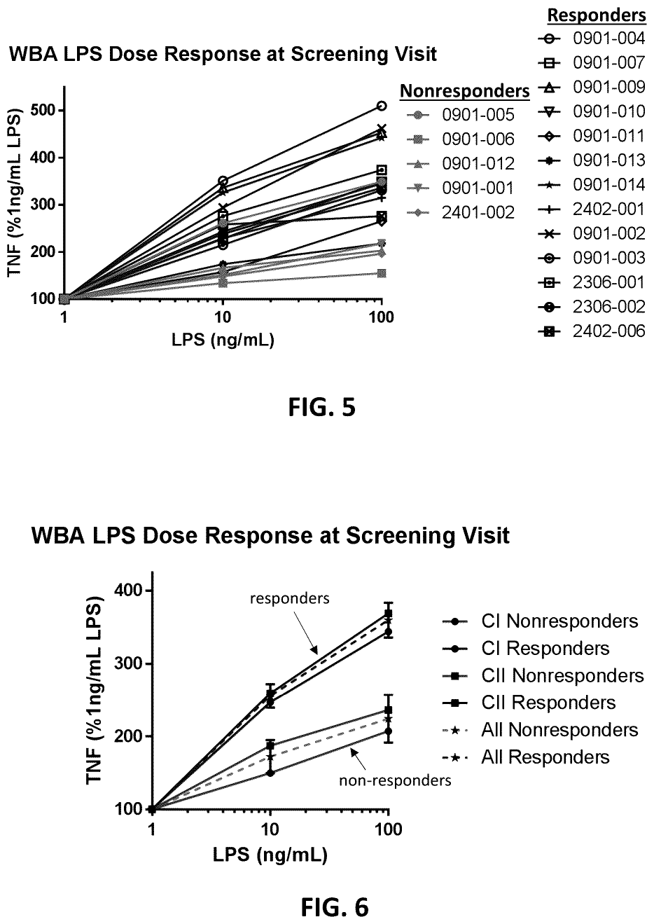

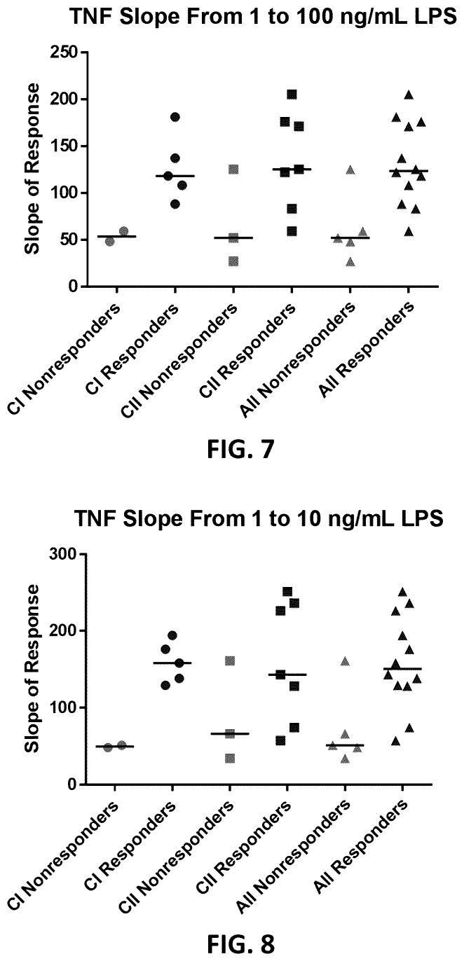

However, a response curve analysis of the TNF level data surprisingly was able to identify and/or separate four out of five nonresponders from the two cohorts of patients (18 patients total—5 nonresponders and 13 responders) with a high level of significance. To perform the response curve analysis, patient-specific TNF levels produced at various LPS concentrations were normalized to TNF levels at 1 ng/mL LPS. Normalized TNF levels produced as a function of LPS concentration was plotted by individual and EULAR response, as shown in FIG. 5, and by cohort and EULAR response, as shown in FIG. 6. Slopes of the response curves were calculated, grouped, and plotted, as shown in FIGS. 7 and 8. For two patients, there was undetectable levels of TNF at 1 ng/mL LPS, so the slopes were extrapolated from the change between 10 and 100 ng/mL LPS.

Based on the data presented in FIG. 7 which presents TNF response data over a 3 log LPS concentration range (1 to 100 ng/mL LPS challenge), 4 of the 5 nonresponders had a slope of less than about 60 while all but one responder had a slope greater than about 60. Similarly, FIG. 8 presents TNF response data over a 2 log LPS concentration range (1 to 10 ng/mL LPS challenge), where 4 out of 5 nonresponders had a slope less than about 75 while all but two responders had a slope greater than about 75.

FIGS. 7 and 8 demonstrate that a screening test based on an assay that measures TNF release or release of another inflammatory cytokine, such as IL-1, IL-6, IL-12, IL-18, inflammsome, interferon gamma, and granulocyte-macrophage colony stimulating factor, from blood cells, particularly white blood cells, in response to LPS/endotoxin challenge over a range of LPS/endotoxin concentrations, can be used to effectively screen patients before implantation to determine whether a candidate is likely to respond or not respond to VNS therapy. In other words, an assay, such as a whole blood assay, that measures a response curve or response over range of challenge concentrations can be used as a screening test. In some embodiments, the range of concentration for the LPS/endotoxin can be at least 2, 3, or 4 logs, where 2 logs means that the highest concentration is 10 times the lowest concentration, and 3 longs means that the highest concentration is 100 times the lowest concentration. In some embodiments, the concentrations may be increased in multiples of at least 2, 3, 4, 5, 6, 7, 8, 9, or 10 times. The screening test can have a cutoff value for the response curve, such as the slope of the response curve or another measure of the degree or magnitude of the response over the concentration range, that establishes whether a candidate is suitable for VNS therapy. In some embodiments, the WBA can be performed using a commercial blood testing kit, such as a TruCulture® kit sold by Myriad RBM. In some embodiments, the blood can be incubated for less than 4 hours, such as less than 3, 2, or 1 hour, or incubation can be greater than 1, 2, 3, or 4 hours. In some embodiments, the assay can use whole blood, or it can use isolated white blood cells or monocytes. In some embodiments, the cells can be stimulated by LPS/endotoxin, while in other embodiments, other stimuli can be used to stimulate the blood cells. In some embodiments, the LPS/endotoxin concentration can range from about 1 ng/mL to about 100 ng/mL. In other embodiments, the low end for the LPS/endotoxin concentration can be less than 1 ng/mL, such as about 100 pg/mL or 10 pg/mL, and the high end can be about 1000 ng/mL or 10000 ng/mL.

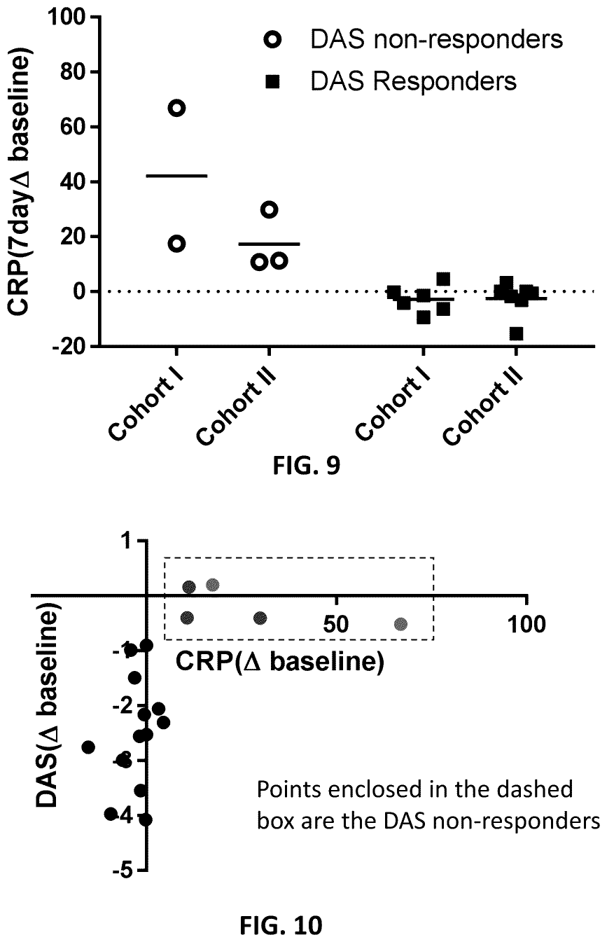

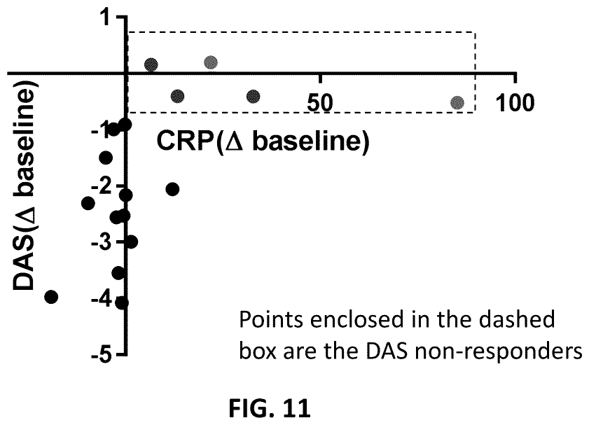

Other tests may be performed after stimulation of the vagus nerve using noninvasive or minimally invasive devices. For example, C-reactive protein (CRP) is a protein synthesized in the liver and secreted into the blood in response to inflammation, particularly in response to IL-6 secretion by macrophages and T-cells. The prospective patient's blood can be collected during the screening day and again after the patient's vagus nerve has been stimulated noninvasively or minimally invasively. After a set or predetermined amount of time after VNS, such as 1, 2, 3, 4, 5, 6, 7, 8, 9, 10, 11, 12, 13, or 14 days, the second blood sample can be collected and both samples can be tested for a biomarker, such as CRP. The first sample taken before VNS stimulation can establish a baseline or reference level of the analyte of interest, and the second blood sample can establish the effect of VNS stimulation on the biomarker. For patients that are likely to respond well to VNS therapy, the CRP levels are likely to remain stable or be reduced, while patients that do not respond well to VNS therapy may show elevated levels of CRP, which may indicate worsening of inflammation. Therefore, patients that do not respond well to VNS therapy may be identified and excluded by setting a cutoff or threshold value based on a comparison of the CRP levels in the second sample to the reference CRP level, as shown in FIGS. 9-11, which show the change in CRP levels at Day 7 (seven days after initial VNS stimulation) with respect to a baseline level for responders and nonresponders (identified at Day 42 using EULAR criteria). For example, the data shown in FIGS. 9-11 indicate that the CRP levels of all the nonresponders increased by at least about 5 to 10 percent relative to the reference CRP level. Therefore, the cutoff for excluding patients can be an increase in CRP levels of greater than 5, 6, 7, 8, 9, 10, 11, 12, 13, 14, or 15 percent after VNS (e.g., non-invasive VNS or acutely invasive VNS).

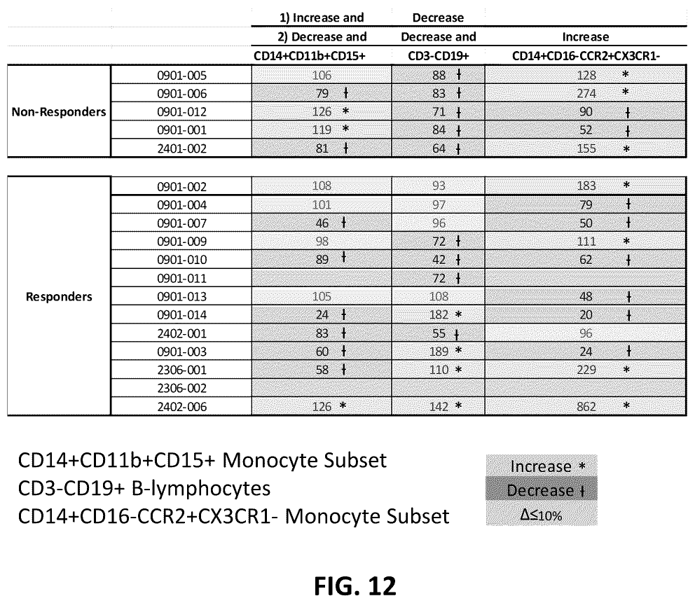

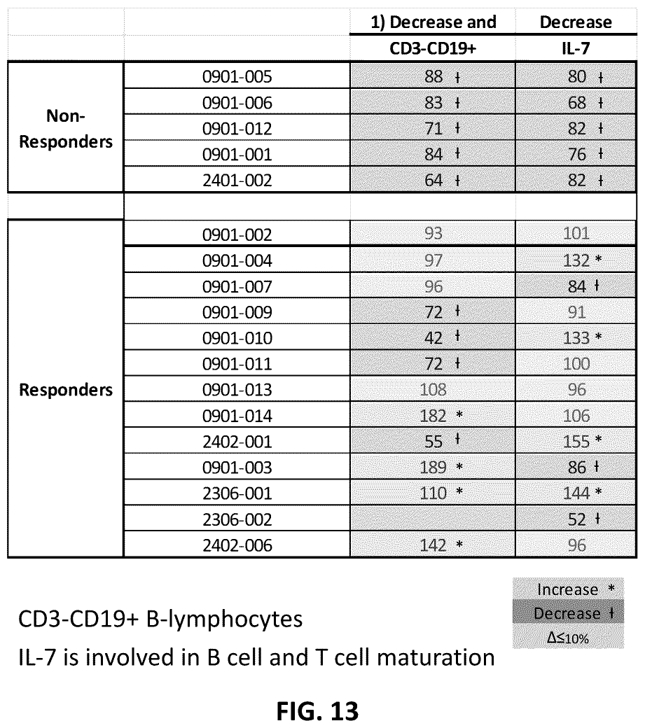

FIGS. 12 and 13 present changes in cell populations and IL-7 levels from D-21 (screening day) to D0 (14 days after implantation of VNS device but before delivering any stimulation). All nonresponders exhibited greater than 10% decrease in CD3−CD19+ (B-lymphocytes) cell populations and greater than 10% decrease in IL-7 levels (see FIG. 13).

Thus a method of treating a patient may include determining if the patient will respond to the treatment based on the relationship of one or more monocyte and lymphocyte populations after a transcutaneous vagus nerve stimulation during screening, particularly in combination with a biomarker such as an inflammatory marker (e.g., IL-7) by comparing the level of the monocytes and/or lymphocytes and the level of the inflammatory biomarker from a baseline taken prior to any treatment with a level following a non-invasive or acutely invasive treatment. In particular, if the level of the monocytes and/or lymphocytes (e.g., CD3−, CD19+B-lymphocytes) and the level of the cytokine (e.g., IL-7) both decrease, the patient may be a non-responder, and implantation may be avoided. In patients for whom either or both the level of monocytes and/or lymphocytes (e.g., CD3−, CD19+B lymphocytes) and the level of the cytokine (e.g., IL-7) increase between baseline and non-invasive or acutely invasive stimulation (which may be performed immediately prior to implantation), implantation may be completed as they are likely to respond to treatment.

### Diagnostic Screening Tests

A diagnostic screening test can predict the likelihood of clinical response to the VNS implant prior to actual implantation surgery (or completion of implantation), for use in clinical decision-making regarding patient selection. Patients that are in need of or that may benefit from a VNS implant can be identified and given the diagnostic screening test. In some embodiments, the test is performed prior to implantation of the device and/or any invasive or noninvasive stimulation of the vagus nerve. In other embodiments, the test involves activating the cholinergic anti-inflammatory pathway for a short duration using techniques that are either non-invasive or minimally invasive as described herein. For example, the vagus nerve can be stimulated electrically or mechanically, as described herein. The extent of the patient's biological response to temporary CAP activation may then be assessed by measuring pathway-mediated inhibition of immune activation in assays performed on blood samples or other biological fluids taken before and after the stimulation. Surprisingly, the extent of the patient's biological response to a brief non- or minimally invasive (e.g., acutely invasive) activation of the pathway can predict clinical response to the permanent implant.

Activation of the cholinergic anti-inflammatory pathway may be achieved through any of the following stimulation techniques: (1) noninvasive transcutaneous stimulation of the auricular branch of the vagus nerve which innervates the skin of the cymba conchae of the ear, using either electrical or mechanical stimulation; (2) stimulation of the cervical vagus nerve using a catheter-directed temporary electrical lead/electrode, introduced intravascularly via percutaneous puncture, and positioned within the cervical internal jugular vein or other nearby vein under fluoroscopic or ultrasound guidance to place it in close apposition to the cervical portion of the vagus nerve; (3) stimulation of the cervical vagus nerve by a temporary catheter-directed non-cuffed electrical lead/electrode, introduced into the carotid sheath by percutaneous puncture, and positioned under fluoroscopic, ultrasound, and/or endoscopic guidance to place it in close apposition to the cervical portion of the vagus nerve within the carotid sheath; (4) stimulation of the cervical vagus nerve by trans-vascular mechanical pressure using an intravascular angioplasty balloon or other expandable element, introduced via percutaneous puncture and positioned under fluoroscopic or ultrasound guidance in close apposition to the cervical portion of the vagus nerve within the cervical internal jugular vein or other nearby vein; and (5) noninvasive transcutaneous cervical vagus nerve stimulation using a transcutaneous electrical nerve stimulation device placed over the vagus nerve on the skin of the patient's neck.

Assessment of the strength of CAP activation can measured using the change from pre- to post stimulation levels in any of the following parameters in a tissue sample or biological fluid such as whole blood, serum, or supernatants from cultures of whole blood, or from cells isolated from whole blood: (1) cytokines or inflammatory mediators or other analytes; and (2) reduction in inducible release of cytokines or other inflammatory mediators or other analytes, from in vitro culture of whole blood or isolated cells from blood. Such induction may be in the form of bacterial lipopolysaccharide, other Toll-like receptor activators, fragments of complement molecules (e.g., C5a), or activating antibodies directed against T cell or B cell surface receptors (e.g. anti CD3/anti CD28 antibodies).

### Alternative Stimulation Locations

Other regions of the subject's body may be alternatively or additionally stimulated, particularly regions enervated by nerves of the inflammatory reflex. For example, the non-invasive stimulation and other stimulation modalities described herein may be applied to the subject's area innervated by the seventh (facial) cranial nerve or cranial nerve V. The non-invasive stimulation and other stimulation modalities described herein may be applied to at least one location selected from: the subject's cymba conchae of the ear, or helix of the ear. In some variations, the non-invasive stimulation and other stimulation modalities described herein is applied to at least one point along the spleen meridian.

It is understood that this disclosure, in many respects, is only illustrative of the numerous alternative device embodiments of the present invention. Changes may be made in the details, particularly in matters of shape, size, material and arrangement of various device components without exceeding the scope of the various embodiments of the invention. Those skilled in the art will appreciate that the exemplary embodiments and descriptions thereof are merely illustrative of the invention as a whole. While several principles of the invention are made clear in the exemplary embodiments described above, those skilled in the art will appreciate that modifications of the structure, arrangement, proportions, elements, materials and methods of use, may be utilized in the practice of the invention, and otherwise, which are particularly adapted to specific environments and operative requirements without departing from the scope of the invention. In addition, while certain features and elements have been described in connection with particular embodiments, those skilled in the art will appreciate that those features and elements can be combined with the other embodiments disclosed herein.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims. The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. An in vitro method for screening a patient for responsiveness to vagus nerve stimulation, the method comprising:
obtaining a sample of blood taken from the patient;
challenging the sample of blood with a toxin;
determining an amount of inflammatory cytokine released in response to the toxin; and
outputting an indication that the patient is a responder or a non-responder for vagus nerve stimulation based on the amount of inflammatory cytokine released from the sample of blood.

2. The method of claim 1, further comprising determining if the patient is a responder for vagus nerve stimulation by determining that a ratio of the amount of the inflammatory cytokine released in response to different concentrations of toxin.

3. The method of claim 1, further comprising implanting a vagus nerve stimulator if the patient is a responder.

4. The method of claim 1, further comprising taking the sample of blood from the patient.

5. The method of claim 1, wherein challenging the sample of blood with the toxin comprises challenging the sample of blood with multiple different concentrations of toxin.

6. The method of claim 1, wherein obtaining the sample of blood taken from the patient prior, comprising obtaining the sample of blood after non-invasively stimulating the patient's vagus nerve.

7. The method of claim 1, wherein the inflammatory cytokine is selected from the group consisting of TNF, IL-1, IL-6, IL-12, IL-18, inflammsome, interferon gamma, and granulocyte-macrophage colony stimulating factor.

8. The method of claim 1, wherein the inflammatory cytokine is TNF.

9. A method for screening a patient for responsiveness to vagus nerve stimulation, the method comprising:
measuring a baseline level of a cytokine and/or C-reactive protein (CRP) in a sample of blood taken from the patient;
stimulating the vagus nerve;
measuring a post-stimulation level of the cytokine and/or CRP in a sample of blood taken after the vagus nerve has been stimulated;
comparing the post-stimulation level of the cytokine and/or CRP to the baseline level of cytokine and/or CRP; and
indicating if the patient is a suitable candidate for an implantable vagus nerve stimulation device based on the comparison of the post-stimulation level of the cytokine and/or CRP to the baseline level of the cytokine and/or CRP.

10. The method of claim 9, further comprising implanting a vagus nerve stimulator if the patient is a suitable candidate.

11. The method of claim 9, wherein indicating comprises indicating that the patient is a suitable candidate for the implantable vagus nerve stimulation device when the post-stimulation level of the cytokine and/or CRP increases by 5% or more.

12. The method of claim 9, wherein the vagus nerve is stimulated noninvasively.

13. The method of claim 9, wherein the vagus nerve is stimulated with transcutaneous electrical stimulation.

14. The method of claim 9, wherein the vagus nerve is stimulated by mechanical stimulation.

15. The method of claim 9, wherein the stimulating the vagus nerve comprises stimulating an auricular branch of the vagus nerve.

16. The method of claim 9, wherein stimulating the vagus nerve comprises stimulating a cervical portion of the vagus nerve.

17. The method of claim 9, further comprising: introducing an electrode intravascularly via percutaneous puncture; and positioning the electrode at a cervically located blood vessel proximate the vagus nerve.

18. The method of claim 9, further comprising: introducing an electrode into a carotid sheath; and positioning the electrode within the carotid sheath such that the electrode is proximate the vagus nerve.

19. The method of claim 9, further comprising: placing an electrode of a transcutaneous electrical nerve stimulation device on the patient's skin over a cervical vagus nerve.

20. The method of claim 9, wherein the cytokine is selected from the group consisting of TNF, IL-1, IL-6, IL-12, IL-18, inflammsome, interferon gamma, and granulocyte-macrophage colony stimulating factor.
